(19) United States
(12) Patent Application Publication
(10) Pub. No.: US 2004/0175358 A1
(43) Pub. Date: Sep. 9, 2004

(54) Enzymes combination coadjuvating in the interferon therapy

(76) Inventors: Raffaele Ansovini, Perugia (IT); Jose Sebastian, Torino (IT)

Correspondence Address: ROTHWELL, FIGG, ERNST & MANBECK, P.C., 1425 K STREET, N.W., SUITE 800, WASHINGTON, DC, 20005, US

(21) Appl. No.: 10/474,433

(22) PCT Filed: Apr. 8, 2002
(86) PCT No.: PCT/EP02/03886

(30) Foreign Application Priority Data
Apr. 10, 2001 (IT) MI01 A 000760

(51) Int. Cl. A61K 38/44; A61K 38/21

(52) U.S. Cl. 424/85.4; 424/94.4; 424/85.6; 424/85.5

(57) ABSTRACT

The use of GSSG-reductase in combination with glutathione peroxidase for the preparation of a medicament adjuvating the therapy with interferons.

The present invention relates to the use of GSSG-reductase in combination with glutathione peroxidase for the preparation of an medicament adjuvating the interferon therapy.

Alpha, beta and gamma interferons, both recombinant and extractive, have been used for some time in human therapy for the treatment of a variety of diseases, mainly of viral or neoplastic type, such as hepatitis B or C, lymphomas, myelomas, carcinomas, Kaposi sarcoma, leukemias, AIDS. One of the main limitations to the therapy with interferons are the side effects which may sometimes be so severe that interruption of the therapy is necessary. The most common side effects include fever, general malaise, leukopenia, myalgia and alopecia.

Therefore, there is a strong need for pharmacological tools which allows to reduce interferons toxicity.

This aim has been reached according to the present invention. The combination of enzymes of the present invention, through complex interactions with the oxidized glutathione (GSSG)/reduced glutathione (GSH) system, not only reduces the side effects of the interferons, but also enhances the activity thereof.

The combination of enzymes of the invention consists of GSSG-reductase and glutathione peroxidase (GPx).

The therapeutical use of the enzyme GSSG-reductase in the treatment of HIV infections has already been disclosed in WO 00/71146. On the other hand, no therapeutical uses of GPx are to date known.

Both these enzymes are commercially available and can be obtained from various sources, such as bacteria, yeasts or with recombinant DNA techniques.

According to the invention, the combination of the two enzymes is administered parenterally, in particular intravenously: one weekly administration of the combination of the invention is usually sufficient during the cycle of therapy with interferons possibly combined with antibiotics, antivirals or chemotherapeutics administration.

The enzymes can be administered at unitary dosages ranging between 100 and 250 mg.

In particular, for interferons dosages above 10 millions units, the enzymes will be preferably administered at doses ranging between 175 and 220 mg. Dosages of 120-170 mg will be preferably administered when the interferons dosage is below 10 millions units. The maximum dose of 250 mg for both enzymes can be administered in case of severe, extreme reactions following therapy with interferons or other drugs, or when higher, repeated doses of such drugs are required.

In the treatment of AIDS, the preferred dose is 160 mg of GSSG-reductase and 110 mg of GPx, independently of the number of interferons units administered.

The enzymes will be formulated in suitable pharmaceutical forms according to conventional techniques: examples of suitable forms comprise sterile, injectable solutions or suspensions. Powder enzymes may optionally be added to saline solutions or to any aqueous solutions for the bolus infusion conventionally used in clinics.

### EXAMPLE

Vials containing 150 mg of GSSG reductase and 150 mg of GPx, both lyophilized, for reconstitution prior to use with a sterile solvent compatible with the intravenous administration.

1. The use of GSSG-reductase in combination with glutathione peroxidase for the preparation of a medicament adjuvating the interferon therapy with interferons, possibly combined with the administration of antibiotics, antivirals and/or chemotherapeutics.

2. The use as claimed in claim 1, wherein interferons are alpha interferon, beta interferons, gamma interferon, of extractive or recombinant origin.

3. The use as claimed in claim 1 or 2, wherein the medicament contains 100 to 250 mg of each enzyme.

4. The use as claimed in any one of claims 1 to 3, wherein the medicament is administered once a week during the cycle of treatment with interferons.
